(19) United States
(12) Patent Application Publication
(10) Pub. No.: US 2007/0198039 A1
(43) Pub. Date: Aug. 23, 2007

(54) Intragastric device for treating obesity

(75) Inventors: Brian K. Jones, Spartanburg, SC (US); Kenneth C. Kennedy II, Clemmons, NC (US); Matthew P. Carter, Dobson, NC (US); Thomas G. Self, Winston-Salem, NC (US); Kimberly K. Ingram, Rural Hall, NC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL, INC., WINSTON-SALEM, NC (US)

(21) Appl. No.: 11/657,231

(22) Filed: Jan. 24, 2007

Related U.S. Application Data
(60) Provisional application No. 60/762,926, filed on Jan. 27, 2006.

(51) Int. Cl. A61B 17/08

(52) U.S. Cl. 606/151

(57) ABSTRACT

An intragastric device generally comprises a strip digestive-resistant mesh material that is operable between a first configuration and a second configuration. The first configuration is sufficiently small to permit introduction of the digestive-resistant mesh material into a gastric lumen of the mammal. The second configuration is sufficiently large to prevent the digestive-resistant mesh material from passing through the mammals pylorus, thereby permitting the mesh member to act as an artificial bezoar. Methods and devices for delivering the mesh member are also provided, including a pusher that is operable to engage and disengage the mesh member for delivery through the gastric lumen via a delivery sheath.

### CROSS-REFERENCE TO RELATED APPLICATIONS

This application claims the benefit of U.S. Provisional Application Ser. No. 60/762,926 filed on Jan. 27, 2006, entitled “INTRAGASTRIC DEVICE FOR TREATING OBESITY”, the entire contents of which are incorporated herein by reference.

### FIELD OF THE INVENTION

This invention relates to medical devices, and more particularly to obesity treatment devices that can be placed in the stomach of a patient to reduce the size of the stomach reservoir.

### BACKGROUND OF THE INVENTION

It is well known that obesity is a very difficult condition to treat. Methods of treatment are varied, and include drugs, behavior therapy, and physical exercise, or often a combinational approach involving two or more of these methods. Unfortunately, results are seldom long term, with many patients eventually returning to their original weight over time. For that reason, obesity, particularly morbid obesity, is often considered an incurable condition. More invasive approaches have been available which have yielded good results in many patients. These include surgical options such as bypass operations or gastroplasty. However, these procedures carry high risks, and are therefore not appropriate for most patients.

In the early 1980s, physicians began to experiment with the placement of intragastric balloons to reduce the size of the stomach reservoir, and consequently its capacity for food. Once deployed in the stomach, the balloon helps to trigger a sensation of fullness and a decreased feeling of hunger. These balloons are typically spherical or pear-shaped, generally range in size from 200-500 ml or more, are made of an elastomer such as silicone, polyurethane, or latex, and are filled with air, water, or saline. While some studies demonstrated modest weight loss, the effects of these balloons often diminished after four to twelve weeks, possibly due to the gradual distension of the stomach or the fact that the body adjusted to the presence of the balloon. Other balloons include a tube exiting the nasal passage that allows the balloon to be periodically deflated and re-insufflated to better simulate normal food intake. However, the disadvantages of having a inflation tube exiting the nose are obvious.

Unrelated to the above-discussed methods for treating obesity, it has been observed that the ingestion of certain indigestible matter, such as fibers, hair, fuzzy materials, etc., can collect in the stomach over time, and eventually form a mass called a bezoar. In some patients, particularly children and the mentally handicapped, bezoars often result from the ingestion of plastic or synthetic materials. In many cases, bezoars can cause indigestion, stomach upset, or vomiting, especially if allowed to grow sufficiently large. It has also been documented that certain individuals having bezoars are subject to weight loss, presumably due to the decrease in the size of the stomach reservoir. Although bezoars may be removed endoscopically, especially in conjunction with a device known as a bezotome or bezotriptor, they, particularly larger ones, often require surgery.

What is needed is an intragastric member that provides the potential weight loss benefits of a bezoar or intragastric balloon while minimizing complications. Ideally, such a-device should be well-tolerated by the patient, effective over a long period of time, sizable for individual anatomies, and easy to place and retrieve.

### BRIEF SUMMARY OF THE INVENTION

The present invention provides an intragastric device for the treatment of obesity in a mammal which acts as an artificial bezoar and is well-tolerated by the stomach. The intragastric device is effective in achieving weight loss over a several month period in animals, while also being easy to place and retrieve. At the same time, the device takes up a smaller volume within the stomach than existing intragastric members such as balloons, with no reduction in efficacy. The intragastric device generally comprises a digestive-resistant mesh material that is operable between a first configuration and a second configuration. The first configuration is sufficiently small to permit introduction of the digestive-resistant mesh material into a gastric lumen of the mammal. The second configuration is sufficiently large to prevent the digestive-resistant mesh material from passing through the mammal's pylorus, thereby permitting the mesh material to act as an artificial bezoar.

According to more detailed aspects of the present invention, the mesh material is elongated in the first configuration and is collected together in the second configuration. The mesh material includes first and second ends, and in the second configuration the first end is positioned near the second end and the mesh material is bundled between the first and second ends. The mesh material is at least partially contained within a delivery sheath in the first configuration, and is delivered through the sheath and collected to form the second configuration. The delivery sheath is sized to be passed through a gastric lumen such as the esophagus.

Another embodiment of intragastric device comprises a yoke and at least one elongated member of digestive-resistant mesh material. The mesh member has a first end and a second end positioned near each other with the mesh material bundled therebetween. The yoke is preferably a thread such as a polypropylene suture, and may be connected to the first and second ends in various manners. Preferably, the thread is tied to the first end of the mesh member and a stopper is used to engage the thread proximate the second end of the strip. The thread is woven through openings in the mesh member, and preferably the mesh member comprises a tube which is temporarily flattened to weave the thread therethrough. The thread may be woven along a centerline of the mesh member or may alternate between opposing sides of the centerline. Likewise, the tube may be folded lengthwise and the thread woven through openings of the mesh material to maintain the folded configuration.

The present invention also provides a method of treating obesity in animals. The method generally includes the introduction of a delivery sheath into a gastric lumen of the mammal, the delivery sheath defining a delivery lumen. A member of digestive-resistant mesh material is introduced into the delivery lumen. The mesh member has a distal end, a proximal end, and a yoke that is connected to the distal end of the mesh member. A pusher is introduced into the delivery lumen and includes a plurality of coupling pawls. The coupling pawls are pointed distally to move the mesh member distally upon distal translation of the pusher. The pusher is reciprocated distally and proximally within the delivery lumen to deliver the mesh member into a stomach of the mammal. The mesh member is bunched into a collected configuration sized to prevent the mesh member from passing through the mammal's pylorus. The yoke is connected to proximal end of the mesh member to maintain the collected configuration thereof.

A delivery device is also provided in accordance with the teachings of the present invention. The delivery device generally comprises a delivery sheath and a pusher. The delivery sheath is sized for introduction into the gastric lumen, and the delivery sheath defines a delivery lumen. The pusher is sized for introduction into the delivery lumen and includes pawls projecting distally. The pawls engage the mesh material when the pusher is moved distally relative to the delivery sheath, and disengage the mesh material when the pusher is moved proximally relative to the delivery sheath.

According to more detailed aspects of the delivery device, the pawls project distally to a free end structured to pass through an opening of the mesh material. The pawls may take many configurations, including being axially spaced and/or circumferentially spaced. Preferably the pawls are formed at a distal end of the pusher. Similarly, the delivery sheath may define retention pawls projecting radially into the delivery lumen. The retention pawls project distally to engage the mesh material when the pusher is moved proximally relative to the delivery sheath. In this manner, the pusher may be reciprocated to advance the mesh member into the stomach while the retention pawls resist proximal movement of the mesh. material. The pusher may take the form of a tube, and the tube may include V-shaped cuts formed therein to define a wedge of tube material that is formed to project radially outwardly and form the pawls. The pusher may also comprise a semi-annular member having a first longitudinal edge and a second longitudinal edge which define the pawls in those edges. The pusher may also take the form of a tube defining a push lumen, and further include a control member positioned inside the pusher lumen and having a plurality of wires projecting distally to form the pawls. The control member and plurality of wires are translatable within the push lumen and operable between a first expanded state for engaging the mesh member and a second withdrawn state for disengaging the mesh member. The plurality of wires are biased radially outwardly towards the first expanded state. In this latter construction, engagement and disengagement of the mesh material may be controlled through the relative translation of the control member and push tube, thereby providing greater control over the reciprocation of the pusher and advancement of the mesh member.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a side view of an intragastric device schematically depicted inside a stomach, constructed in accordance with the teachings of the present invention;

FIG. 2 is a perspective view of the intragastric device of FIG. 1, depicted in an elongated configuration;

FIG. 3 is a perspective view of the intragastric device of FIG. 1, depicted in an intermediate configuration;

FIG. 4 is a perspective view of the intragastric device of FIG. 1, depicted in a collected configuration;

FIG. 5 is a partial perspective view depicting the formation of the intragastric device of FIG. 1;

FIG. 6 is a partial perspective view depicting the formation of an intragastric device constructed in accordance with the teachings of the present invention;

FIG. 7 is a partial perspective view depicting the formation of an intragastric device constructed in accordance with the teachings of the present invention;

FIG. 8 is a cross-sectional view of a delivery device for introducing the intragastric device of FIG. 1 into the stomach;

FIG. 9 is a side view of a pusher tube forming a part of the delivery device depicted in FIG. 8;

FIG. 10 is a side view of an alternate embodiment of the pusher device depicted in FIG. 9;

FIG. 11 is a cross-sectional view showing another embodiment of a delivery device constructed in accordance with the teachings of the present;

FIG. 12 is a cross-sectional view showing another embodiment of a delivery device constructed in accordance with the teachings of the present invention;

FIG. 13 is another cross-sectional view of the delivery device depicted in FIG. 12;

FIG. 14 is a side view, partially cut-away, of another embodiment of a delivery device constructed in accordance with the teachings of the present invention;

FIG. 15 is a plan view, partially cut-away, of a mesh member forming part of an intragastric device;

FIG. 16 is a side view of the mesh member of FIG. 15; and

FIG. 17 is a perspective view of another embodiment of a mesh member forming part of an intragastric device.

### DETAILED DESCRIPTION OF THE INVENTION

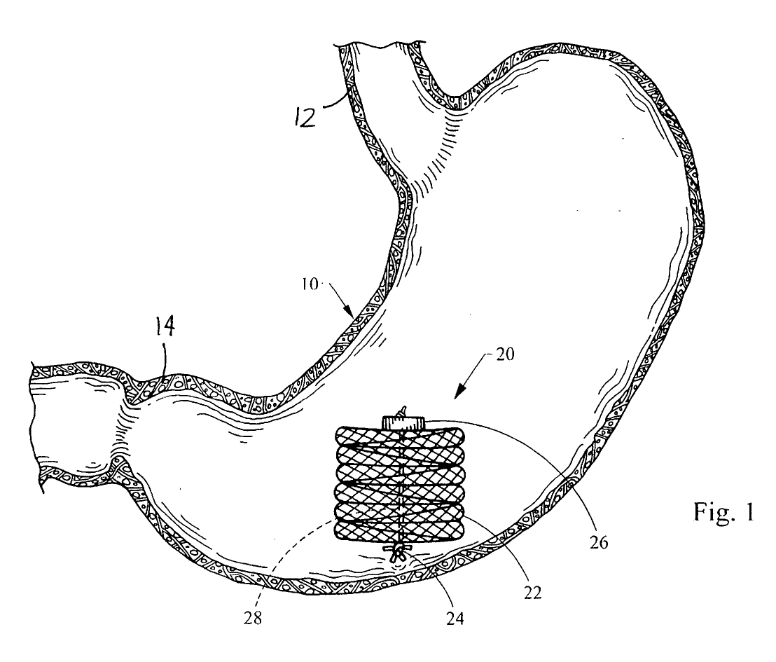

Turning now to the figures, FIGS. 1-4 depict an intragastric device 20 for treating obesity, constructed in accordance with the teachings of the present invention. One or more intragastric devices 20 may be used to act as an artificial bezoar to achieve weight loss. The intragastric device 20 is introduced through a gastrointestinal lumen of a mammalian patient, such as the esophagus 12, and passed into the stomach 10 to reside therein, generally being unable to pass through the pylorus 14.

The intragastric device 20 comprises one or more digestive-resistant or indigestible members 22. As used herein, the terms digestive-resistant and indigestible are intended to mean that the material used is not subject to the degrative effects of stomach acid and enzymes, or the general environment found within the gastric system over an extended period of time, therefore allowing the device to remain intact for the intended life of the device. This does not necessarily mean that the material cannot be degraded over time; however, one skilled in medical arts and gastrointestinal devices would readily appreciate the range of material that would be suitable for use as a long-term intragastric member.

Many well-known plastics have suitable properties for forming the indigestible member 22, including selected polyesters, polyurethanes, polyethylenes, polytetrafluoroethylene (PTFE), polyamides, silicone, or other possible materials. Mammalian hair has been found to form natural bezoars, and thus, is also a possible material. However, some materials, such as certain polyamides, have been found to expand over time, which can be an undesirable property. Most other natural materials are generally much less resistant to acids and enzymes, and would therefore typically require treatment or combination with resistant materials to function long term, unless a shorter-term placement is intended or desired.

The indigestible member 22 is also constructed from a mesh or mesh material, and may take many forms including strips, tubes, and sheets, although many other forms will be readily apparent to those skilled in the art. A tubular mesh member 22 has been depicted in the figures and will be used to describe the invention, although any of these forms may be readily employed. Notably, it is believed that the use of a mesh material for the indigestible member 22 improves patient tolerance of the intragastric device 20. More particularly, the pliability and feel of the mesh material reduces the potential for trauma to the stomach lining, thereby improving tolerance. Likewise, the mesh material is very easily bent, twisted and folded, simplifying delivery and removal while easily conforming to the interior of the stomach 10.

As used herein, “mesh” or “mesh material” refers to an open material, fabric or structure having a plurality of spaced apart openings. Many types of mesh materials may be employed, including interwoven strands 23 (like a fabric) of indigestible materials (one woven example being depicted in FIGS. 15 and 16), composites such as filter materials, or indigestible materials formed as a mesh through the creation of spaced apart openings 25 (one example depicted in FIG. 17). For example, a strip or tube could be molded to include such spaced apart openings 25. Preferably, the mesh material is formed by strands interconnected at intervals, and most preferably by plastic strands bonded at regular intervals, as depicted by the mesh member 22 of FIGS. 1-4. In the preferred form, the interconnected strands are formed by a thermoplastic such as medium density polyethylene (MDPE).

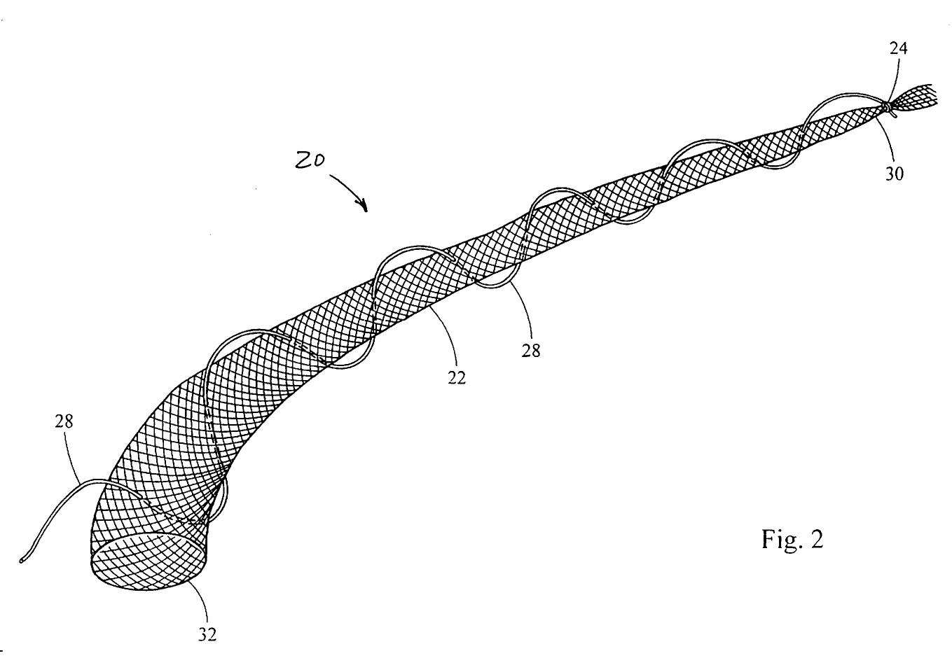

The intragastric device 20 is operable between a first elongated configuration, shown in FIG. 2, and a second collected configuration depicted in FIGS. 1 and 4. In the first elongated configuration, the mesh member 22 may be passed through the esophagus 12 and into the stomach, as will be described in more detail later herein. In the first configuration, the mesh member 22 is generally elongated between a first distal end 30 and a second proximal end 32. In the second collected configuration, the mesh member 22 resides in the stomach 10 and acts as an artificial bezoar. In the collected configuration, the intragastric device generally includes a first button 24 and a second button 26 connected to a yoke 28 to bundle the mesh member 22 between the buttons 24, 26. As used herein, “bundle” and “bundled” refers to the mesh material being folded, crunched, looped or otherwise longitudinally collected. As also used herein, “between” does not require that all of the material of the mesh member 22 be entirely positioned between the first and second buttons 24, 26 in the second collected configuration (the opposite being clearly seen in the figures), but rather simply refers to the material in-between the first and second ends of the elongated mesh member.

The yoke 28 is woven through the mesh member 22 at regular intervals between the distal end 30 and the proximal end 32. The yoke 28 may take many forms, but preferably comprises a thread such as a polypropylene suture, although many suture or thread materials may be employed. Other forms of the yoke 28 include structures such as plastic tubing or solid rods. A first end 30 of the mesh member 22 is closed, preferably by tying the suture 28 around the mesh tube to define a first button 24. It will be recognized by those skilled in the art that the first end 30 may be closed, and likewise the suture 28 connected to the first end 30, in numerous manners. For example, the plastic material of the mesh member 22 may be heated and melted to form the first button 24. The suture 28 may be then tied to the first end 30 and/or button 24, or the first end 30 may be melted around the suture 28. Likewise, the first end 30 may be tied in a knot to define the button 24. A separate stopper or other clamp may also form the button 24. Many variations for the button 24 and for connecting the suture 28 to the first end 30 will be readily apparent to those skilled in the art.

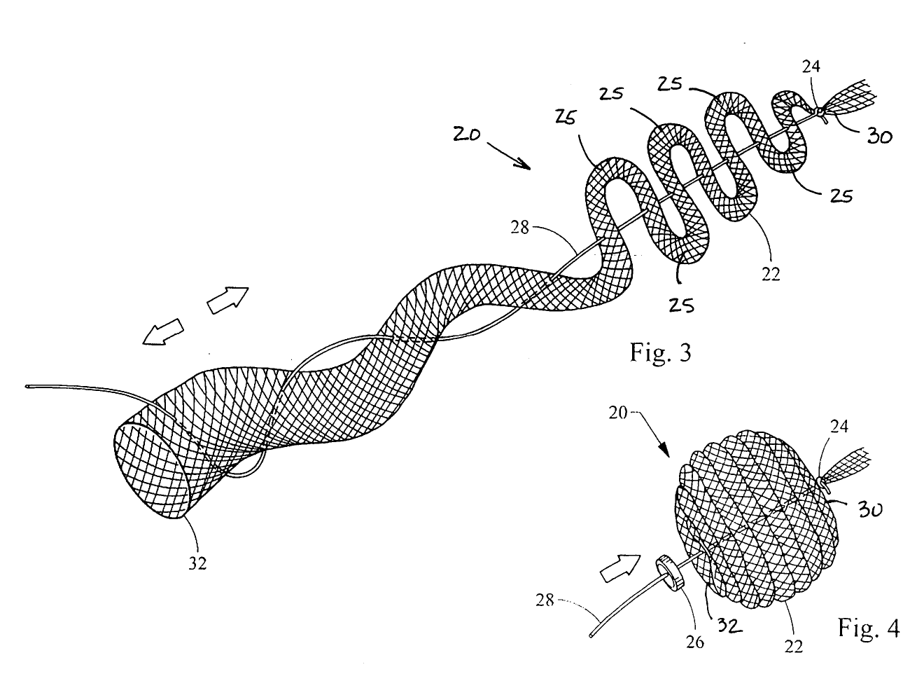

The transition of the mesh member 22 from the first configuration to the second collected configuration is depicted with reference to FIGS. 3 and 4. Generally, distal translation of the mesh member 22 over the suture 28 results in the collection of mesh member 22, which folds on itself back and forth, to define loops 25. As seen in FIG. 3, by connecting the suture 28 to the first end and weaving the suture 28 through the mesh material, the mesh member 22 may be bundled at the first end 30. As the second proximal end 32 continues to be advanced over the yoke 28 towards the first end 30, the collected mesh member 22 forms a ball-like mass as seen in FIG. 4. The second end 32 is moved distally to a point near the first end 30, and is maintained in this position by a second button 26. Preferably, the second button 26 is a plastic stopper fitted over the suture 28 and is slid distally to engage the second end 32. The resulting intragastric device 20 is shown in FIG. 1 residing in the stomach 10 and acting as an artificial bezoar.

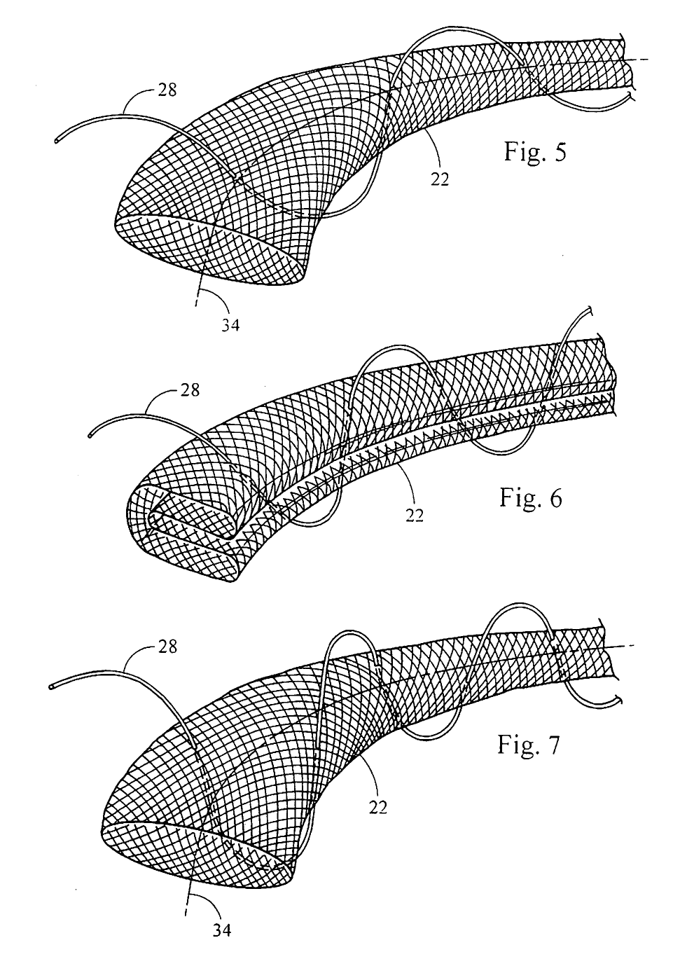

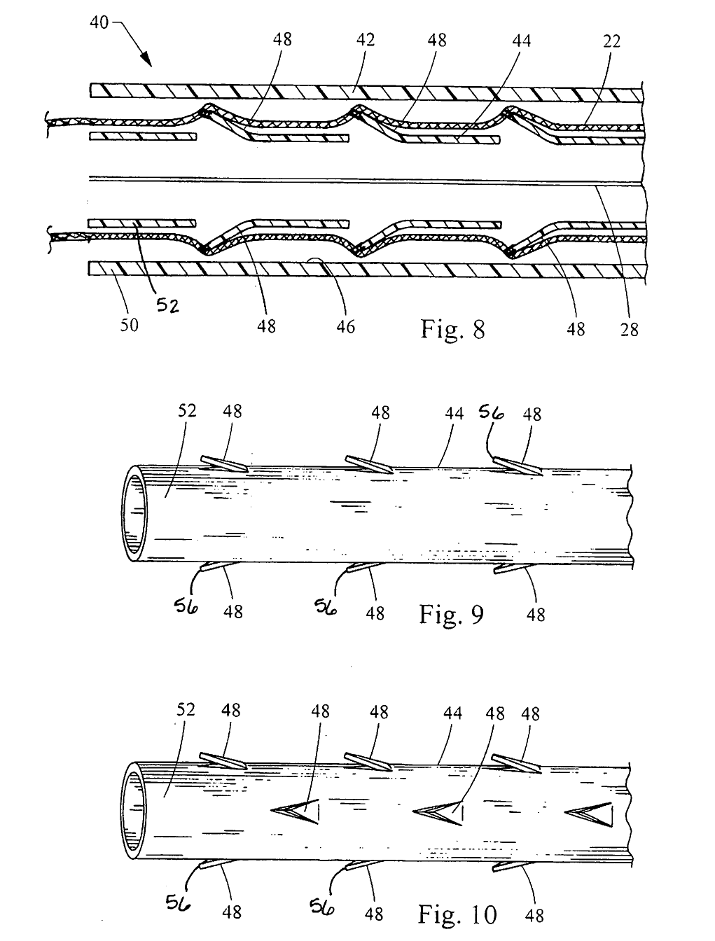

The suture 28 may be woven through the mesh member 22 in many different manners, and a few preferred configurations are depicted in FIGS. 5-7. As depicted in FIG. 5, the mesh tube 22 is temporarily flattened and generally defines a centerline 34 extending longitudinally therethrough. The suture 28 is woven through the mesh member 22 along the centerline 34 at regularly spaced intervals. Preferably the intervals are about 5 inches, although this distance may be varied depending on the size of the mesh member 22, and the desired size and shape of the intragastric device 20. FIG. 6 depicts the mesh member 22 temporarily flattened and folded lengthwise. The suture 28 is then woven through all layers of the folded mesh member 22 to maintain the folded configuration. FIG. 7 depicts the mesh member 22 being temporarily flattened to define a longitudinal centerline 34. In this embodiment, the suture 28 is woven through the mesh member 22 on alternating sides of the centerline 34. As will be discussed further below, FIG. 8 illustrates that the suture 28 may simply extend through the interior of the tubular mesh member 22, so long as it is connected to the first and second ends 30, 32 thereof.

It will be recognized by those skilled in the art that numerous configurations of the intragastric member 20 may be realized through variation of the size of the mesh member 22, the mesh material forming the mesh member 22, the type of yoke 28 employed, as well as the passing of the yoke 28 through the mesh member 22 following a pathway that is infinitely variable. For example, the mesh member 22 may not be tubular but may simply comprise a flat strip of material of constant or varying width, and the suture 28 may be woven along an irregular path. Additional examples of such variations of the intragastric device 20 are disclosed in copending U.S. patent application Ser. No. 10/151,720 filed May 27, 2002 and U.S. Patent Application 60/679,135 filed May 9, 2005, the disclosures of which are hereby incorporated by reference in their entirety.

In the first elongated configuration, the mesh member 22 generally has a length to width ratio that is about 400 to about 1000 for passage into the stomach 10, while in the second collected configuration the mesh member 22 generally has a length to width ratio that is less that about 5, and preferably about 1 (i.e. an about equal length and width) for residence in the stomach 10. As one example, a mesh member that has a length of 20 feet (uncollected), and a diameter of about 0.5 inches (diameter being considered about equal to the width), would have a length to width ratio of about 480 in the first elongated configuration.

Deployment of intragastric device 20 can be accomplished in a number of ways, depending on the size, number, and configuration of the devices, or according to physician or patient preference. Turning now to FIGS. 8-14, several embodiments of delivery devices and methods for delivering the intragastric device 20 will be described. With reference to FIGS. 8 and 9, the delivery device 40 generally includes a delivery sheath 42 and a pusher 44. The delivery sheath 42 is formed as a cannula or other tubular member, and preferably is constructed of a flexible plastic material, and most preferably polyvinylchloride (PVC) formed over a stainless steel coil or other reinforcing member. The delivery sheath 42 defines a delivery lumen 46, the delivery sheath 42 being sized to be introduced through a gastrointestinal lumen (such as the esophagus 12) of the mammalian patient. The mesh member 22 is advanced past a distal end 50 of the delivery sheath 42 and into the stomach 10.

The pusher 44 is also constructed from a cannula or other tubular member. The pusher 44 defines a plurality of coupling pawls 48 which are structured for engagement and translation of the mesh member 22 through the delivery lumen 46 of the delivery tube 42. As best seen in FIG. 9, the pusher 44 includes a plurality of pawls 48 formed at a distal end 52 thereof which are structured to engage the mesh member 22 when moved distally relative to the delivery sheath 42, but disengage the mesh member 22 when moved proximally relative to the delivery sheath 42. By the term “disengage”, it will be recognized by those skilled in the art that the pawls 48 will contact the mesh member 22 when moved proximally, but due to the construction of the pawls 48, the mesh member 22 will not be substantially moved in the proximal direction. The pawls 48 project distally from the pusher tube 44 to a pointed distal end 56. Further, the pawls 48 project radially outward from the pusher tube 44. Due to this construction, the pawls 48 act as a one-way engagement mechanism, whereby distal translation of the pusher tube 44 will cause the free ends 56 of the pawls 48 to pass through holes formed in the mesh member 22 and translate the mesh member 22 distally. Conversely, proximal translation of the pusher tube 44 generally results in the withdrawal of the pawls 48 and their distal ends 56 from the openings in the mesh member 22, to minimize or eliminate any proximal translation of the mesh member 22. The pusher tube 44 may be constructed of numerous materials, preferably a flexible plastic, one preferred material being a low density polyethylene (LDPE) which provides sufficient radial flexibility to the pusher 44 while also having sufficient axial rigidity to provide the one-way engagement function of the pawls 48 and distal translation of the mesh member 22.

In one preferred construction, the delivery sheath 42 has an inner diameter of 0.65 inches, while an outer diameter of the pusher tube 44 (measured to the distal tip 56 of the pawls 48) is approximately 0.50 inches. In a relaxed state, the mesh member 22 has a diameter of about 2 inches, resulting in substantial folding or overlap of the mesh member 22 within the delivery sheath 42. Preferably, the pawls 48 extend axially about 0.25 inch and are angled about 15-45 degrees (from a central axis of the pusher tube 44). The pawls 48 are unitarily formed with the pusher tube 44, typically by making a V-shaped cut in the material and manipulating the pawls 48 into their distally and radially outwardly projecting configuration.

It can be seen from FIG. 8 that the pusher tube 44 extends within the interior of the tubular mesh member 22. That is, the tubular mesh member 22 extends between the outer diameter of the pusher tube 44 and the inner diameter of the delivery sheath 42. Accordingly, it can be seen the reciprocal axial motion of the pusher tube 44 within the delivery sheath 42 results in corresponding engagement and disengagement of the pawls 48 with the mesh member 22 and distal translation of the mesh member 22 into the stomach 10 of the mammal. It can also be seen in the embodiment of FIG. 8 that the yoke 28, depicted in the figure as a polypropylene suture, extends axially through the center of the mesh member 22 without being woven through the openings of the mesh material. Accordingly, this configuration results in a more random bunching of the mesh member 22 in the second collected configuration (FIG. 1).

It will be recognized by those skilled in the art that the coupling pawls 48 of the pusher tube 44 may take numerous relative configurations. In the embodiment depicted in FIGS. 8-9, the coupling pawls are arranged in three series of pawls 48 (the series being depicted as pairs of pawls 48), the three series being axially spaced apart. In each series, the pawls 48 are circumferentially spaced. At the same time, corresponding pawls 48 of each series are circumferentially aligned. As one example, the pawls 48 of the axially-spaced series may not be circumferentially aligned. In FIG. 10, adjacent series of pawls 48 are circumferentially spaced apart (i.e. non-aligned) to provide greater coverage around the periphery of the pusher tube 44.

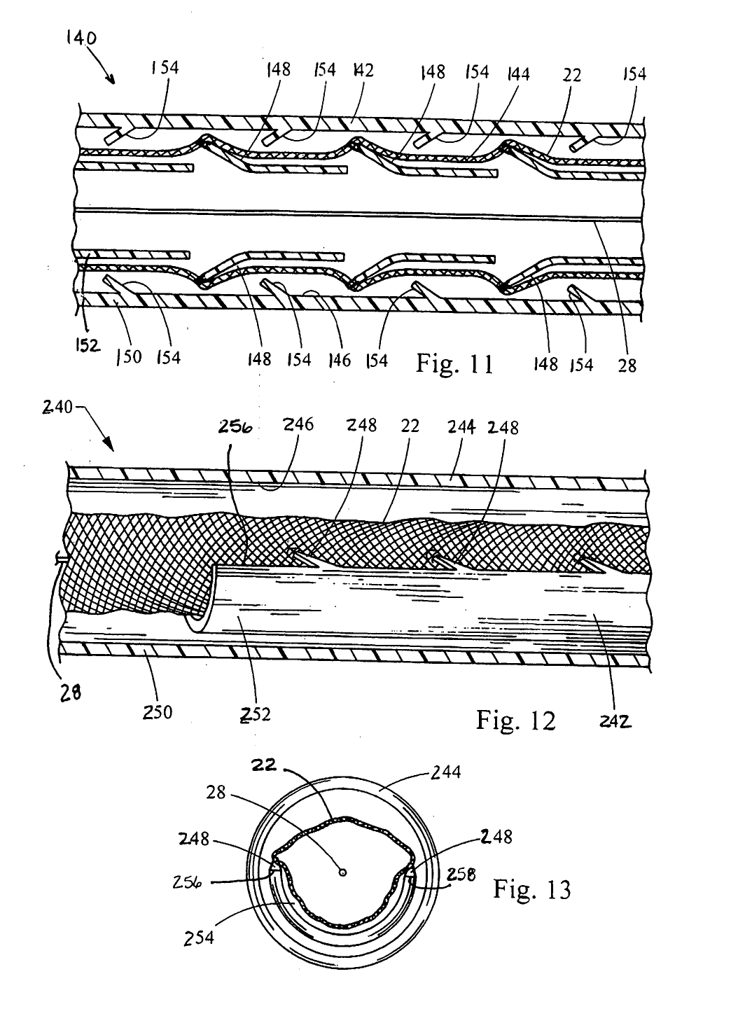

An alternate embodiment of the delivery device 140 may utilize an alternately constructed delivery sheath 142 which assists in resisting the proximal motion of the mesh member 22 therein. As shown in FIG. 11, the delivery sheath 142 defines a delivery lumen 146 receiving the mesh member 22 and a pusher 144. The pusher 144 again includes a plurality of pawls 148 for engaging and disengaging the mesh member 22 through reciprocal motion of the pusher 144 relative to the delivery sheath 142. However, in this embodiment the delivery sheath 142 includes retention pawls 154 projecting radially inwardly into the delivery lumen 146. The retention pawls 154 also extend distally, whereby the retention pawls engage, the mesh member 22 when the pusher 144 is moved proximally relative to the delivery sheath 142, but disengage the mesh member 22 when the pusher 144 is moved distally relative to the delivery sheath 142.

Turning now to FIG. 12, another alternate embodiment of the delivery device 240 has been depicted in accordance with the teachings of the present invention. In this embodiment, the delivery device 240 again includes a tubular delivery sheath 242 constructed similar to the prior embodiment to define a delivery lumen 246 receiving the tubular mesh material 22 for delivery through a distal end 250 of the delivery sheath 242. However, in this embodiment the pusher 244 has a semi-annular shape as best seen in FIG. 13. Stated another way, the pusher 244 may be formed of a cannula or other tubular member which is cut lengthwise such that the pusher 244 defines a radially opening inner surface 254, a first longitudinally extending edge 256 and a second longitudinally extending edge 258.

This construction also permits the pawls 248 to be unitarily formed with the pusher 244. That is, the edges 256, 258 may be shaped to form the pawls 248 at a distal end 252 of the pusher tube 244. The pawls 248 extend distally to a free end which is structured to pass through the openings in the mesh member 22. The pawls 248 are also radially aligned with the pusher 244 along the first and second longitudinally extending edges 256, 258, although the pawls 248 may project radially inward for engagement of the mesh member 22. Unlike the prior embodiment, the mesh member 22 is positioned inside the pusher 244, and extends along the inner surface 254 thereof. Reciprocal motion of the pusher 244 distally and proximally will cause the pawls 248 to engage and disengage, respectively, the mesh member 22 for advancement into the stomach 10 of the mammal.

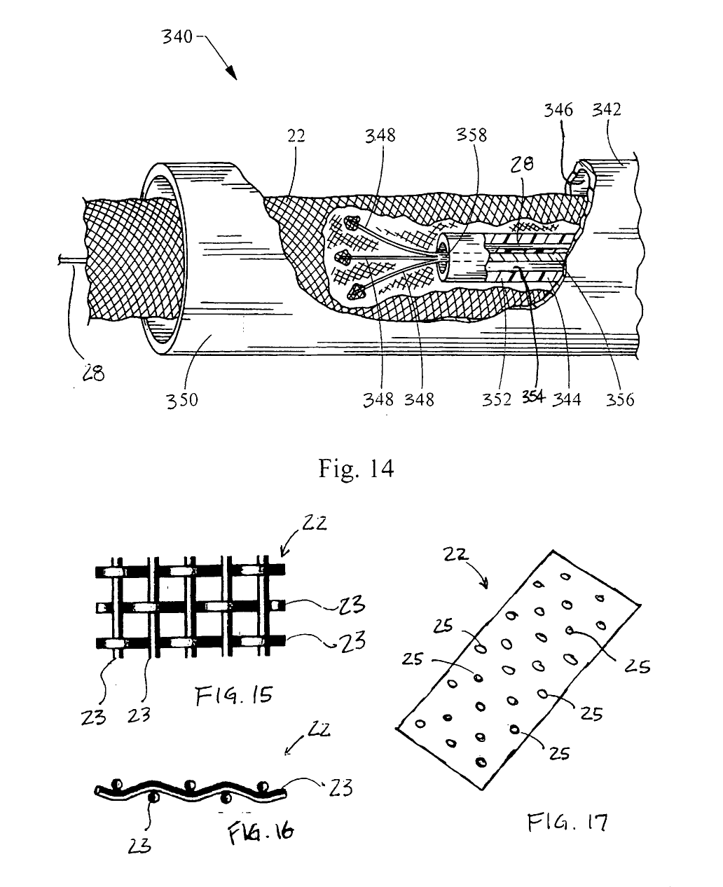

Yet another embodiment of the delivery device 340 has been depicted in FIG. 14. As with the prior embodiments, the delivery device 340 includes a delivery sheath 342 defining a delivery lumen 346 receiving a pusher 344. In this embodiment, the pusher 344 is a tubular member defining an inner lumen 354 and having a distal end 352 receiving a control member 356. The control member 356 may comprise a wire or a plastic rod permitting translation of the control member 356 relative to the pusher tube 344. Additionally, the coupling pawls are formed by a plurality of wires 348 which are connected to the control member 356 at a hub 358, such as by soldering, welding or other well known techniques. The pawl wires 348 project distally, and are biased in the radially outward direction. Preferably, the pawl wires 348 are formed of a memory material such as a superelastic alloy, and preferably nitinol (a nickel-titanium alloy) to provide this outward bias.

It will therefore be recognized that the translation of the control member 356 and pawl wires 348 relative to the pusher tube 344 permits the pawl wires 348 to be withdrawn inside the pusher lumen 354, or to project distally from the end 352 of pusher 344. Thus, the pawl wires 348 are operable between a first expanded state as depicted in FIG. 14, wherein the pawl wires 348 are positioned to extend through the openings in the mesh member 22 and engage the strip for distal translation. However, when the control member 356 and pawl wires 348 are translated proximally relative to the pusher tube 344, the pawl wires 348 take a second withdrawn state wherein the pawl wires 348 are disengaged from the mesh member 22. In this manner, the pawl wires 348 may take their first expanded state and the pusher tube 344 and control member 356 moved together distally to advance the mesh member 22 out of the distal end 350 of the delivery sheath 342. The control member 356 may then be operated to cause the pawl wires 348 to take their withdrawn state, disengaging the mesh member 22, and permitting the pusher tube 344 and control member to be moved together proximally, the process repeated to deliver the mesh member 22 into the stomach 10 of the mammal.

It will also be recognized by those skilled in the art that in the embodiment of FIG. 14, the pawl wires 348 need not be withdrawn to disengage the mesh member 22. That is, the structure of the pawl wires 348 results in the natural engagement and disengagement of the mesh member 22 through distal and proximal translation relative to delivery sheath 342, respectively, as was the case with prior embodiments. Accordingly, the pusher tube 352 can be dispensed with (or the pusher wires 348 attached directly to the pusher 352 to form a control member 356).

The present invention also encompasses a method of treating obesity in mammals utilizing the mesh member 22 and delivery device 40, 140, 240, 340 to form an intragastric device 20 acting as an artificial bezoar. The method will be described with reference to the delivery device 40, but is equally applicable to all the delivery devices. Generally, the method includes the introduction of the delivery sheath 42, into the gastric lumen 12 of the mammal, the delivery sheath 42 defining the delivery lumen 46. The strip of digestive-resistant mesh material 22 is introduced into the delivery lumen 46. The mesh member 22 has distal end 30, proximal end 32, and a yoke 28 is connected to the distal 30 end of the mesh member 22. A pusher 44 is introduced into the delivery lumen 46 and includes a plurality of coupling pawls 48. The coupling pawls 48 are pointed distally to move the mesh member 22 distally upon distal translation of the pusher 44. The pusher 44 is reciprocated distally and proximally within the delivery lumen 46 to deliver the mesh member 22 into a stomach of the mammal. The mesh member 22 is bunched into a collected configuration sized to prevent the mesh member 22 from passing through the mammal's pylorus 14. The yoke 28 is connected to the proximal end 32 of the mesh member 22 to maintain the collected configuration thereof. Once the insertion and collection procedures are completed, any excess yoke (i.e. suture) 28 is removed.

To retrieve the intragastric device 20 and its mesh member 22, the mesh member 22 is returned from its collected configuration (FIG. 4) to its elongated configuration (FIG. 3). To achieve the change in configurations, the buttons 24, 26 may be removed, and/or the yoke 28 may be cut, thereby freeing the material of the mesh member 22. The above-described steps are generally reverse, to un-collect or un-spool the mesh member 22 and return it to its elongated configuration. Of course, this configuration transition may occur over time as the mesh member is withdrawn from the stomach 10 through the esophagus 12, leaving a portion of the mesh member 22 collect within the stomach 10. The buttons 24, 26 and yoke 28 may be removed, such as by using grasping forceps or other tool, or alternatively may be allowed to pass naturally through the digestive system.

Accordingly, it will be recognized by those skilled in the art that the present invention provides an intragastric device, a delivery device therefore, and a method of treating obesity in mammals which is easy to place and retrieve. Ideally, the intragastric device acts as an artificial bezoar to achieve weight loss. At the same time, the present invention can potentially take up a smaller volume within the stomach than existing intragastric members such as balloons.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An intragastric device for the treatment of obesity in a mammal, the intragastric device comprising:
a digestive-resistant mesh material that is operable between a first configuration and a second configuration, the first configuration being sufficiently small to permit introduction of the digestive-resistant mesh material into a gastric lumen of the mammal, the second configuration being sufficiently large to prevent the digestive-resistant mesh material from passing through the mammal's pylorus and to act as an artificial bezoar.

2. The intragastric device of claim 1, wherein the mesh material is an elongated strip in the first configuration.

3. The intragastric device of claim 1, wherein the mesh material is collected together in the second configuration.

4. The intragastric device of claim 2, wherein the elongated strip includes first and second ends, and in the second configuration the first end is positioned near the second end and the mesh material is bundled between the first and second ends.

5. The intragastric device of claim 1, wherein the mesh material is tubular.

6. The intragastric device of claim 1, wherein the mesh material is at least partially contained within a delivery sheath when in the first configuration, and is removed from the delivery sheath and bundled to form the second configuration.

7. The intragastric device of claim 6, wherein the delivery sheath is sized to be passed through a gastrointestinal lumen.

8. The intragastric device of claim 1, further comprising a yoke maintaining the collected mesh material in the second configuration.

9. The intragastric device of claim 8, wherein the yoke is a thread.

10. The intragastric device of claim 1, wherein the first configuration of the digestive-resistant mesh material has a length to width ratio of about 300 to about 1000.

11. The intragastric device of claim 1, wherein the second configuration of the digestive-resistant mesh material has a length to width ratio of less than about 5.

12. An intragastric device for the treatment of obesity in a mammal, the intragastric device comprising:
a member of digestive-resistant mesh material having a first end and a second end, the first end positioned in near the second end, the mesh member bundled between the first and second ends; and
a yoke connected to the first end and to the second end to maintain the position of the first end near the second end.

13. The intragastric device of claim 12, wherein the mesh member is a tube of the digestive-resistant mesh material.

14. The intragastric device of claim 13, wherein the first end of the tube is closed.

15. The intragastric device of claim 13, wherein the tube is temporarily flattened and folded lengthwise and the yoke is woven through openings of the mesh material to maintain the folded configuration.

16. The intragastric device of claim 13, wherein the tube is temporarily flattened to define a centerline, and wherein the thread is woven in a manner alternating between opposing sides of the centerline.

17. The intragastric device of claim 13, wherein the tube is temporarily flattened to define a centerline, and wherein the thread is woven along the centerline in regularly spaced intervals.

18. The intragastric device of claim 12, wherein the yoke is a thread.

19. The intragastric device of claim 18, wherein the thread is tied to the first end of the mesh member.

20. The intragastric device of claim 18, further comprising a stopper engaging the thread proximate the second end of the mesh member.

21. The intragastric device of claim 18, wherein the thread is woven through openings of the mesh material.

22. The intragastric device of claim 18, wherein the mesh member is bundled in a configuration sufficiently large to prevent the intragastric device from passing through the mammal's pylorus.

23. A method of treating obesity in mammals, the method comprising the steps of:
introducing a delivery sheath into a gastric lumen of the mammal, the delivery sheath defining a delivery lumen;
introducing a member of digestive-resistant mesh material into the delivery lumen, the mesh member having a distal end, a proximal end, and having a yoke connected to the distal end;
introducing a pusher into the delivery lumen, the pusher including a plurality of coupling pawls, the coupling pawls pointed distally to move the mesh member distally upon distal translation of the pusher;
reciprocating the pusher distally and proximally within the delivery lumen to deliver the mesh member into a stomach of the mammal;
bunching the mesh member into a collected configuration sized to prevent the mesh member from passing through the mammal's pylorus; and
connecting the yoke to the proximal end of the mesh member to maintain the collected configuration of the mesh member.

24. The method of claim 23, wherein the mesh member is tubular and defines a strip lumen.

25. The method of claim 24, wherein the pusher is introduced inside the strip lumen.

26. The method of claim 23, wherein the pusher is introduced between the delivery sheath and the mesh member.

27. The method of claim 23, wherein the step of connecting the yoke to the proximal end includes connecting a button to the yoke and translating the button distally.

28. The method of claim 23, wherein the coupling pawls engage the mesh member when moved distally, and disengage the mesh member when moved proximally.

29. A delivery device for delivering an elongated strip of mesh material into a stomach of a mammal via a gastric lumen, the delivery device comprising:
a delivery sheath sized for introduction into the gastric lumen, the delivery sheath defining a delivery lumen; and
a pusher sized for introduction into the delivery lumen, the pusher having pawls projecting distally, the pawls engaging the mesh material when the pusher is moved distally relative to the delivery sheath, the pawls disengaging the mesh material when the pusher is moved proximally relative to the delivery sheath.

30. The delivery device of claim 29, wherein the pawls project distally to a free end structured to pass through an opening of the mesh material.

31. The delivery device of claim 29, wherein the pawls are axially spaced.

32. The delivery device of claim 29, wherein the pawls are circumferentially spaced.

33. The delivery device of claim 29, wherein the pawls are formed at a distal end of the pusher.

34. The delivery device of claim 29, wherein the delivery sheath defines retention pawls projecting radially inwardly into the delivery lumen.

35. The delivery device of claim 34, wherein the retention pawls project distally to engage the mesh material when the pusher is moved proximally relative to the delivery sheath.

36. The delivery device of claim 29, wherein the pusher is a tube, the tube being sized to be received inside the delivery lumen.

37. The delivery device of claim 36, wherein the pawls project radially outwardly.

38. The delivery device of claim 36, wherein the pawls are unitarily formed from the tube.

39. The delivery device of claim 38, wherein the tube includes V-shaped cuts formed therein to define a wedge of tube material that is formed to project radially outwardly to form the pawls.

40. The delivery device of claim 29, wherein the pusher is a semi-annular member having a first longitudinally extending edge and a second longitudinally extending edge.

41. The delivery device of claim 40, wherein at least one of the first or second longitudinally extending edges defines the pawls.

42. The delivery device of claim 29, wherein the pusher is a tube defining a pusher lumen, and further includes a control member positioned inside the pusher lumen, the control member having a plurality of wires projecting distally to form the pawls.

43. The delivery device of claim 42, wherein the control member and plurality of wires are translatable within the pusher lumen and operable between a first expanded state for engaging the mesh member and a second withdrawn state for disengaging the mesh member.

44. The delivery device of claim 43, wherein the plurality of wires are biased radially outwardly towards the first expanded state.
